(19) United States
(12) Patent Application Publication
(10) Pub. No.: US 2022/0211640 A1
(43) Pub. Date: Jul. 7, 2022

(54) MEDICINAL COMPOSITIONS OF CANNABINOIDS AND XANTHINES

(71) Applicant: JHO INTELLECTUAL PROPERTY HOLDINGS, LLC, Weston, FL (US)

(72) Inventors: John H. Owoc, Southwest Ranches, FL (US); Liangxi Li, Plantation, FL (US)

(21) Appl. No.: 17/699,947

(22) Filed: Mar. 21, 2022

Related U.S. Application Data
(63) Continuation-in-part of application No. 16/541,731, filed on Aug. 15, 2019, now Pat. No. 11,278,516.
Application No. 16/541,731 is a continuation-in-part of application No. 16/541,770, filed on Aug. 15, 2019.

(51) Int. Cl. A61K 31/05; A61K 31/522; A61K 36/35; A61K 9/00; A61K 36/31; A61K 31/352; A61K 36/185; A61K 31/353; A23L 33/105; A23L 2/52

(52) U.S. Cl. CPC A61K 31/05; A61K 31/522; A61K 36/35; A61K 9/0095; A61K 36/31; A23V 2002/00; A61K 36/185; A61K 31/353; A23L 33/105; A23L 2/52; A61K 31/352

(57) ABSTRACT

Various embodiments relate to bioactive compositions, including a first component including 1 to 300 mg/dose of a psychotropic cannabinoid, a non-psychotropic cannabinoid, or a mixture thereof; a second component including 1 to 400 mg/dose of a methylxanthine; and an aqueous carrier.

### CROSS-REFERENCE TO RELATED APPLICATIONS

This application is:
a continuation-in-part application of U.S. application Ser. No. 16/541,731, filed on Aug. 15, 2019; and
a continuation-in-part application of U.S. application Ser. No. 16/541,770, filed on Aug. 15, 2019.

The disclosure of each prior application is incorporated by reference into the present application in its entirety.

### TECHNICAL FIELD

The present disclosure relates to nutritional, nutraceutical, and/or medicinal compositions containing a psychotropic cannabinoid, a non-psychotropic cannabinoid, or a mixture thereof; and a xanthine. The present disclosure further relates to compositions containing a bioavailable cannabinoids in combination with xanthines.

### BACKGROUND

Cannabinoids are chemical compounds found in the cannabis plant that interact with receptors in the brain and body to create various effects. Herbal cannabis contains over 100 cannabinoids unique to the plant genus Cannabis. The pharmacology of most of the cannabinoids is largely unknown but the most potent psychoactive agent, Δ⁹-tetrahydrocannabinol (Δ⁹-THC, or THC), has been isolated, synthesized and much studied due to its abundance and psychoactive attributes. Other plant cannabinoids include cannabinol (CBN) and cannabidiol (CBD). These and other cannabinoids have entourage effects with THC and may modify its actions when herbal cannabis is smoked.

The most notable cannabinoids include tetrahydrocannabinol (Δ⁹-THC), cannabidiol (CBD) and cannabinol (CBN). Notable plant-derived non-psychotropic cannabinoids include cannabinol (CBN), cannabidiol (CBD), cannabigerol (CBG), tetrahydrocannabivarin (THCV), and cannabichromine (CBC). Recent evidence shows that non-psychotropic cannabinoids counteracts cognitive impairment associated with the use of cannabis. Most cannabinoids derive from cannabigerol-type compounds and differ mainly in the way this precursor is cyclized. The classical cannabinoids are derived from their respective 2-carboxylic acids (2-COOH) by decarboxylation (catalyzed by heat, light, or alkaline conditions).

Like most pharmacologically active secondary metabolites of plants, THC is a lipid found in cannabis, assumed to be involved in the plant's self-defense, putatively against insect predation, ultraviolet light, and environmental stress.

The actions of THC result from its partial agonist activity at the cannabinoid receptor CB₁ (Kᵢ=10 nM) located mainly in the central nervous system, and the CB₂ receptor (Kᵢ=24 nM), mainly expressed in cells of the immune system. The psychoactive effects of THC are primarily mediated by the activation of cannabinoid receptors, which result in a decrease in the concentration of the second messenger molecule cAMP through inhibition of adenylate cyclase.

Cannabidiol has low affinity for the cannabinoid CB₁ and CB₂ receptors, although it can act as an antagonist of CB₁/CB₂ agonists despite this low affinity. Cannabidiol may be an antagonist of GPR55, a G protein-coupled receptor and putative cannabinoid receptor that is expressed in the caudate nucleus and putamen in the brain. It also may act as an inverse agonist of GPR3, GPR6, and GPR12. CBD has been shown to act as a serotonin 5-HT_{1A} receptor partial agonist and this action may be involved in its antidepressant, anxiolytic, and neuroprotective effects. It is an allosteric modulator of the .mu.- and .delta.-opioid receptors as well. The pharmacological effects of CBD may involve PPARγ agonism and intracellular calcium release.

Cannabinol (CBN) is a mildly psychoactive cannabinoid found only in trace amounts in Cannabis and is mostly found in aged Cannabis. Pharmacologically relevant quantities are formed as a metabolite of tetrahydrocannabinol (THC). CBN acts as a partial agonist at the CB₁ receptors but has a higher affinity to CB₂ receptors; however, it has lower affinities relative to THC. Degraded or oxidized cannabis products, such as low-quality baled cannabis and traditionally produced hashish, are high in CBN.

Selective breeding of cannabis plants has expanded and diversified as commercial and therapeutic markets develop. Some growers in the US succeeded in lowering the proportion of CBD-to-THC to accommodate customers who preferred varietals that were more mind-altering due to the higher THC and lower CBD content.

It is well known that cannabinoids, especially CBD and THC have many medicinal benefits to treat several medical and psychological conditions. Cannabinoids may be used to treat epilepsy, anxiety, sleep disorders, (alertness at low doses and sedation at high doses), psychosis and movement disorders, relief of neuropathic pain in patients with multiple sclerosis, emesis, reduce food intake, anti-proliferative/pro-apoptotic effects, and antibacterial activity, anti-inflammatory, psoriasis, analgesic activity, and anti-spasmodic activity. Cannabinoids may also be used to treat Post Traumatic Stress Disorder, neuropathic and chronic pain, insomnia, nausea, inflammation, arthritis, migraines, Cancer, Crohn's disease, fibromyalgia, Alzheimer's disease, Multiple sclerosis, Glaucoma, Attention deficit hyperactivity disorder (“ADHD”), sleep apnea and appetite loss. CBD's subtle effects are primarily felt in pain, inflammation, and anxiety relief, as well as other medicinal benefits. CBD also does not have any adverse side effects that may occur with consumption of THC. Unlike THC, CBD also does not cause a high. CBD also appears to counteract the sleep-inducing effects of THC, which may explain why some strains of cannabis are known to increase alertness. CBD also acts to reduce the intoxicating effects of THC, such as memory impairment and paranoia. There is also pre-clinical evidence to warrant clinical studies into their use for the treatment of diabetes, ischemia, neurodegeneration, cancer, chronic liver disease and obesity.

Similar to THC, CBN also activates the endocannabinoid system (ECS) to produce its therapeutic effects. In addition to this, like CBD, CBN also stimulates other non-cannabinoid receptors as well. These receptors play a part in modulating several symptoms that contribute to the worsening of anxiety symptoms.

Caffeine is a central nervous system (CNS) stimulant of the methylxanthine class. It is the most widely consumed psychoactive drug. Unlike many other psychoactive substances, it is legal and unregulated in nearly all parts of the world. There are several known mechanisms of action to explain the effects of caffeine. The most prominent is that it reversibly blocks the action of adenosine on its receptor and consequently prevents the onset of drowsiness induced by adenosine. Caffeine also stimulates certain portions of the autonomic nervous system. In addition, caffeine induces ketosis in mammals. At normal doses, caffeine has variable effects on learning and memory, but it generally improves reaction time, wakefulness, concentration, and motor coordination. Caffeine is a proven ergogenic aid in humans. Caffeine improves athletic performance in aerobic (especially endurance sports) and anaerobic conditions. Moderate doses of caffeine (around 5 mg/kg) can improve sprint performance, cycling and running time trial performance, endurance (i.e., it delays the onset of muscle fatigue and central fatigue), and cycling power output. Caffeine increases basal metabolic rate in adults. Caffeine improves muscular strength and power and may enhance muscular endurance. Caffeine also enhances performance on anaerobic tests. Caffeine consumption before constant load exercise is associated with reduced perceived exertion. While this effect is not present during to exhaustion exercise, performance is significantly enhanced. This is congruent with caffeine reducing perceived exertion, because exercise to exhaustion should end at the same point of fatigue. Caffeine also improves power output and reduces time to completion in aerobic time trials, an effect positively (but not exclusively) associated with longer duration exercise.

Although many health benefits can be derived from caffeine, it can produce anxiety and mild form of drug dependence—associated with withdrawal symptoms such as sleepiness, headache, and irritability—when an individual stops using caffeine after repeated daily intake. Tolerance to the autonomic effects of increased blood pressure and heart rate, and increased urine output, develops with chronic use (i.e., these symptoms become less pronounced or do not occur following consistent use).

Nootropics (colloquial: smart drugs and cognitive enhancers) are drugs, supplements, and other substances that may improve cognitive function, particularly executive functions, memory, creativity, or motivation, in healthy individuals.

### SUMMARY

In light of the present need for products with reduced side effects associated with caffeine, a brief summary of various embodiments is presented. Some simplifications and omissions may be made in the following summary, which is intended to highlight and introduce some aspects of the embodiments, but not to limit the scope of the invention. Detailed descriptions of an exemplary embodiment adequate to allow those of ordinary skill in the art to make and use the inventive concepts will follow in later sections.

The present invention provides medicinal compositions containing cannabinoids and caffeine that are bioavailable.

Various embodiments disclosed herein relate to a nutritional or nutraceutical composition, comprising:
a first component comprising 1 to 300 mg/dose of a psychotropic cannabinoid, a non-psychotropic cannabinoid, or a mixture thereof;
a second component comprising 1 to 400 mg/dose of a methylxanthine; and\ an aqueous carrier;
wherein the aqueous carrier is not derived from a tea or coffee.

In various embodiments, the first component is selected from the group consisting of Δ9-tetrahydrocannabinol (Δ9-THC), cannabidiol (CBD), cannabigerol (CBG), Cannabinol (CBN), cannabichromene (CBC), cannabidivarin (CBDV), and mixtures thereof. The methylxanthine may be selected from the group consisting of caffeine, aminophylline, dyphylline, 3-isobutyl-1 methylxanthine, paraxanthine, pentoxifylline, theobromine, theophylline, and mixtures thereof. The composition may further include an extract of valerian containing a valerenic acid, which may be valerenic acid, hydroxyvalerenic acid, acetoxyvalerenic acid, or a mixture thereof

In various embodiments, the nutritional or nutraceutical composition is in the form of an orally administrable liquid composition. The orally administrable liquid composition may be an aqueous solution, a fruit juice, yogurt, or a pudding. The orally administrable liquid composition may be a dispersion or suspension of a powder in an aqueous medium.

Various embodiments disclosed herein relate to a nutraceutical composition, including:
a first component comprising 1 to 50 mg/dose of a psychotropic cannabinoid, a non-psychotropic cannabinoid, or a mixture thereof;
a second component comprising 80 to 300 mg/dose of a methylxanthine; and
an aqueous carrier,
   wherein the first component is free of non-cannabinoid active components of Cannabis sattiva and the second component contains no active components of coffee other than the methylxanthine.
      The first component and the second component may be used in a ratio from 1:30 to 5:8, or from 1:5 to 1:12.

Various embodiments disclosed herein relate to a treatment method including:
administering an effective amount of a nutraceutical composition to a mammal in need thereof,
wherein the nutraceutical composition comprises a first component comprising 1 to 300 mg/dose of a psychotropic cannabinoid, a non-psychotropic cannabinoid, or a mixture thereof; and a second component comprising 1 to 400 mg/dose of a methylxanthine; and thereby
imparting a nootropic effect beyond effects obtained by cannabinoids alone. The method may involve administering the nutraceutical composition once a day over a period of treatment of one to eight weeks.

Various embodiments disclosed herein relate to a treatment method including:
administering an effective amount of the nutraceutical composition to a mammal in need thereof, wherein the nutraceutical composition includes:
   a first component comprising 1 to 50 mg/dose of a psychotropic cannabinoid, a non-psychotropic cannabinoid, or a mixture thereof; and
   a second component comprising 80 to 300 mg/dose of a methylxanthine;
   wherein the first component and the second component are used in a ratio from 1:30 to 5:8; and thereby
imparting a nootropic effect beyond effects obtained by cannabinoids alone. The method may involve administering the nutraceutical composition once a day over a period of treatment of one to eight weeks.

The treatment methods disclosed herein may include, prior to administration, providing the nutraceutical composition in a powder form; and dispersing or suspending the composition in an aqueous vehicle or carrier to form a liquid composition.

### DETAILED DESCRIPTION

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for describing particular embodiments only and is not intended to be limiting.

The articles “a” and “an” are used herein to refer to one or to more than one (i.e., to at least one) of the grammatical object of the article. By way of example, “an element” means one element or more than one element. Thus, recitation of “a composition”, for example, includes a plurality of the compositions. The term “about,” as used to modify numbers or ranges, means±10%, ±5%, or ±2%.

The term “cannabinoid,” as used herein, may refer to the terpenophenolic constituents of Cannabis sativa. The word “cannabinoid,” as used herein, may also refer to a chemical substance, regardless of structure or origin, which binds the cannabinoid receptors of the body and brain. The bicyclic sesquiterpene, β-caryophyllene, frequently found in plants including Cannabis sativa, has been shown to selectively target the CB2 receptor, and may therefore be considered as a cannabinoid herein. In various preferred embodiments, the word “cannabinoid” may also refer to naturally occurring compounds, including terpenophenolic compounds, which bind cannabinoid receptors.

The terpenophenolic constituents of Cannabis sativa, commonly described as cannabinoids, include Δ⁹-tetrahydrocannabinol (Δ⁹-THC, formula I), cannabidiol (CBD, formula II), cannabigerol (CBG, formula III), Cannabinol (CBN, formula IV), cannabichromene (CBC, formula V) and cannabidivarin (CBDV, formula VI). Psychotropic cannabinoids include Δ⁹-THC, also known as THC. As understood herein, non-psychotropic cannabinoids include CBD, CBG, CBN, CBC, and CBDV.

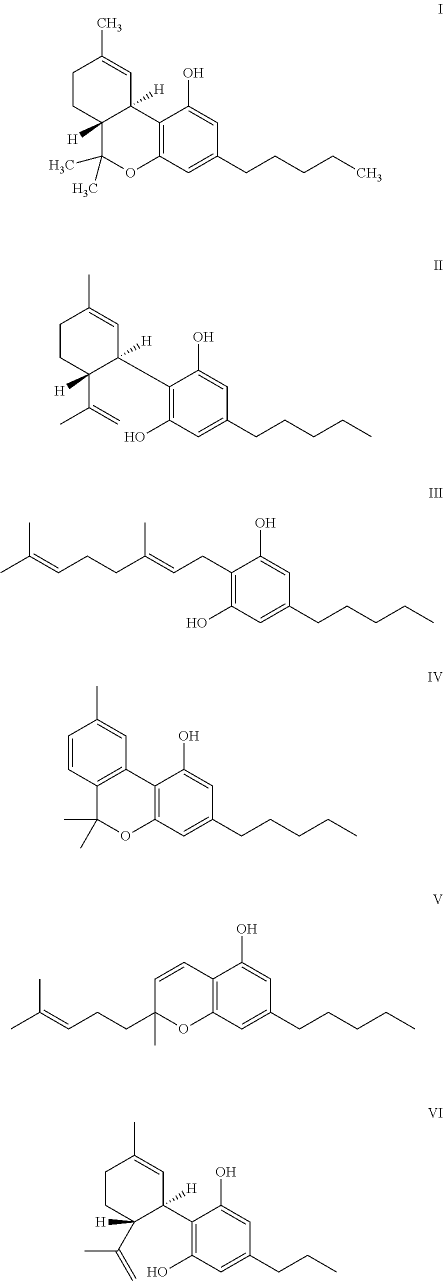

As understood herein, a “non-psychotropic” compound has <25%, <15%, <10%, or <5% of the psychotropic activity of THC.

As understood herein, xanthine refers to a purine-derived group of pharmacologic agents and includes, but is not limited to, methylated xanthines (methylxanthines), which include, but are not limited to caffeine, aminophylline, dyphylline, IBMX (3-isobutyl-1-methylxanthine), paraxanthine, pentoxifylline, theobromine, theophylline, and pharmaceutically acceptable salts thereof.

Yohimbine derivatives include Yohimbine HCl, Yohimbine Monoglycine ester, Yohimbine Alkyl Amine, or combinations thereof.

In one embodiment, the present invention is novel in that it is formed to provide blends of cannabinoids with xanthine classes of compounds as medicinal dosage forms that are bioavailable, fast acting and highly metabolized, with consistent results that take place in a consistent amount of time.

In another embodiment, the present invention is novel in that it is formed to provide blends of cannabinoids with xanthine classes of compounds which provide a nootropic effect beyond the effects simple obtained by cannabinoids alone.

In another embodiment, the present invention is novel in that it is formed to provide blends of cannabinoids with xanthine classes of compounds which reduces anxiety due to ingestion of xanthines containing beverages.

In one embodiment, the present invention provides blends of non-psychotropic cannabinoids with xanthine classes of compounds as nutraceutical supplement that are bioavailable, fast acting and highly metabolized, with consistent results that take place in a consistent amount of time. As understood herein, Xanthine refers to all xanthines and includes, but is not limited to methylated xanthines (methylxanthines), which include, but are not limited to caffeine, aminophylline, IBMX (3-isobutyl-1-methylxanthine), paraxanthine, pentoxifylline, theobromine, and theophylline.

In another embodiment, the present invention provides blends of non-psychotropic cannabinoids with xanthine classes of compounds which provide a nootropic effect beyond the effects simple obtained by cannabinoids alone.

In another embodiment, the present invention provides blends of non-psychotropic cannabinoids with xanthine classes of compounds which reduces anxiety due to ingestion of xanthines containing beverages. Similarly reduces the side effects associated with the exogenous ingestion of yohimbine and its derivatives.

In another embodiment, the present invention provides blends of non-psychotropic cannabinoids with xanthine classes of compounds which amplify alertness and decrease attention deficit disorders by improving cognitive function, reaction time, focus, energy, and psychomotor vigilance.

In one embodiment, the present invention provides blends of:

A psychotropic cannabinoid, a non-psychotropic cannabinoid, or a mixture thereof,

A xanthine compound, and optionally

Yohimbine or a derivative thereof, and optionally

Valerian extract comprising valerenic acid compounds.

The blend is used as a nutraceutical or pharmaceutical supplement that is bioavailable, fast acting, and highly metabolized, with consistent results that take place in a consistent amount of time.

In another embodiment, the present invention provides blends of non-psychotropic or psychotropic cannabinoids with xanthine classes of compounds which provide a nootropic effect beyond the effects simple obtained by cannabinoids alone.

In another embodiment, the present invention provides blends of non-psychotropic or psychotropic cannabinoids with a xanthine compound which reduce anxiety due to ingestion of xanthine-containing beverages. Similarly reduces the side effects associated with the exogenous ingestion of yohimbine and its derivatives.

In another embodiment, the present invention provides blends of non-psychotropic or psychotropic cannabinoids with a xanthine compound which amplify alertness and decrease attention deficit disorders by improving cognitive function, reaction time, focus, energy, and psychomotor vigilance.

In another embodiment, the present invention provides blends of non-psychotropic or psychotropic cannabinoids with a xanthine compounds which reduce the side effects associated with the exogenous ingestion of yohimbine and its derivatives.

In another embodiment, the present invention is novel in that it is formed to provide blends of cannabinoids with xanthine classes of compounds which amplify alertness and decrease attention deficit disorders by improving cognitive function, reaction time, focus, energy, and psychomotor vigilance.

In one embodiment, the medicinal composition is in the form of a powder composition. This medicinal composition includes:

tetrahydrocannabinol (THC), cannabidiol (CBD), or cannabinol (CBN),

Caffeine or any other xanthine derivative; and optionally

Yohimbine or a derivative.

In an exemplary embodiment, the composition is in the form of a powder. An exemplary serving size of this powder composition is from 10 to 1000 mg of active ingredients, said active ingredients being THC; CBD; CBN, or any other cannabinoid or any constituent of hemp; caffeine or any other xanthine; or combinations thereof.

Optionally, the composition may include from 1 to 30 mg of yohimbine or any of its derivatives.

In various embodiments, the composition comprises a dose wherein:

a total content of the first active component and the second active component is between 2 mg and 700 mg per dose, wherein:

the first active component is a cannabinoid, e.g., CBD or THC, present in 1-300 mg/dose, 1-100 mg/dose, 1-50 mg/dose, 2-250 mg/dose, 5-200 mg/dose, 10-150 mg/dose, 10-100 mg/dose, 10-50 mg/dose, 20-75 mg/dose, 20-50 mg/dose, or 25-30 mg/dose, and

the second active component is a xanthine, e.g., a methylxanthine, e.g., caffeine, present in 1-400 mg/dose, 5-350 mg/dose, 10-300 mg/dose, 20-200 mg/dose, 30-300 mg/dose, 40-250 mg/dose, 50-400 mg/dose, 50-350 mg/dose, 50-300 mg/dose, 75-300 mg/dose, 80-300 mg/dose, 100-300 mg/dose, 150-275 mg/dose, or 200-250 mg/dose, as seen in Table 1.

The composition may comprise a dose wherein:

a total content of the first active component and the second active component is between 20 mg and 400 mg per dose, wherein:

the first active component is a cannabinoid, e.g., CBD or THC, present in 10-100 mg/dose, 10-50 mg/dose, 20-50 mg/dose, or 25-30 mg/dose, and

the second active component is a xanthine, e.g., a methylxanthine, e.g., caffeine, present in 10-300 mg/dose, 80-300 mg/dose, 100-300 mg/dose, 150-275 mg/dose, or 200-250 mg/dose.

The composition may comprise a dose wherein:

a total content of the first active component and the second active component is between 20 mg and 400 mg per dose, wherein:

the first active component is a cannabinoid present in 10-100 mg/dose, 10-50 mg/dose, 20-50 mg/dose, or 25-30 mg/dose, where the cannabinoid is:
the psychotropic cannabinoid THC;
a non-psychotropic cannabinoid selected from CBD, CBG, CBN, CBC, CBDV, or a mixture thereof; or
a mixture of THC and a non-psychotropic cannabinoid in a ratio of from 1:100 to 10:1, from 1:50 to 5:1, from 1:30 to 1:1, from 1:25 to 1:5, from 1:10 to 1:5, from 1:20 to 1:10, or from 1:18 to 1:13; and

the second active component is a methylxanthine present in 10-300 mg/dose, 80-300 mg/dose, 100-300 mg/dose, 150-300 mg/dose, or 200-250 mg/dose, where the methylxanthine is selected from caffeine, aminophylline, dyphylline, 3-isobutyl-1methylxanthine, paraxanthine, pentoxifylline, theobromine, theophylline, and mixtures thereof. In various embodiments, the cannabinoid and the methylxanthine are used in a ratio of from 1:30 to 10:1, 1:30 to 5:8, 1:15 to 1:2, 1:5 to 1:12, or 1:7 to 1:10.

In various embodiments, the cannabinoid and the methylxanthine are provided in an aqueous vehicle, which may be a carbonated or non-carbonated aqueous vehicle flavored with natural or artificial flavors; natural sweeteners, e.g., sugar, fructose, honey, allulose, mogrosides, steviosides, sugar alcohols, etc.; and/or artificial sweeteners, e.g., Acesulfame potassium, aspartame, cyclamate, saccharin, sucralose, etc.

In various embodiments, the aqueous vehicle is not derived from coffee or tea, where a tea is derived from steeping herbal ingredients in general, or Camellia sinensis in particular, in water. In various embodiments, the aqueous vehicle is free of:

non-cannabinoid active ingredients found in Cannabis sativa; and/or

active ingredients normally found in coffee or Camellia sinensis other than methylxanthines. Thus, the formulation does not include active ingredients normally found in coffee, such as chlorogenic acids, quinic acid, 3,5-dicaffeoylquinic acid, and diterpenes. Additionally, since the aqueous vehicle is not a coffee- or tea-based composition, it may contain reduced levels of compounds found in tea or coffee, e.g., polyphenols, which introduce a bitter or astringent taste to the formulation. In various embodiments, the formulation may be free of polyphenols.

The aqueous vehicle may be a carbonated or noncarbonated aqueous carrier containing natural or artificial sweeteners and/or natural or artificial flavors.

The composition may comprise a dose containing:

from 2-50 mg/dose of the psychotropic cannabinoid THC; a non-psychotropic cannabinoid; or a mixture of THC and a non-psychotropic cannabinoid; and

a methylxanthine selected from caffeine, aminophylline, dyphylline, 3-isobutyl-1methylxanthine, paraxanthine, pentoxifylline, theobromine, theophylline, and mixtures thereof,

where the cannabinoid and the methylxanthine are used in a ratio of from 1:7 to 1:10.

In various embodiments, the composition may comprise:
from 2-20 mg/dose, from 3-15 mg/dose, or from 4-10 mg/dose of the psychotropic cannabinoid THC;
from 10-50 mg/dose, from 15-40 mg/dose, or from 20-30 mg/dose of the non-psychotropic cannabinoid;
a mixture of from 2-10 mg/dose of THC; and from 0-48 mg/dose of the non-psychotropic cannabinoid;
a mixture of from 3-8 mg/dose of THC; and from 8-40 mg/dose of the non-psychotropic cannabinoid;
a mixture of from 4-6 mg/dose of THC; and from 16-35 mg/dose of the non-psychotropic cannabinoid; or
a mixture of about 5 mg/dose of THC; and 20-25 mg/dose of the non-psychotropic cannabinoid; and

the methylxanthine.

The composition may comprise a dose wherein:

a first active component is a cannabinoid, e.g., CBD or THC, present in 1-100 mg/dose, 1-50 mg/dose, 2-50 mg/dose, or 25-30 mg/dose;

a second active component is a xanthine, e.g., a methylxanthine, e.g., caffeine, present in 10-300 mg/dose, 80-300 mg/dose, 100-300 mg/dose, 150-275 mg/dose, or 200-250 mg/dose; and

a third active ingredient, e.g., a valerian extract comprising from 2 to 3 mg/g of valerenic acids, e.g., a valerian extract comprising from 2 to 3 mg/g extract of valerenic acid, hydroxyvalerenic acid, and/or acetoxyvalerenic acid.

In various embodiments, the formulation is a nutritional composition, where the cannabinoid may be provided as a hemp extract, obtained from:

hemp containing <0.3% THC and ˜5.0% CBD; and/or

hemp containing THC and a non-psychotropic cannabinoid in a ratio of about

In some embodiments, the formulation is a nutraceutical or pharmaceutical composition, where the cannabinoid comprises, per dose, 1.5 to 25 mg, 2.5 to 15 mg, or 4 to 10 mg THC and 10 to 75 mg, 15 to 50 mg, or 20 to 25 mg of a non-psychotropic cannabinoid. The cannabinoid may comprise 5 mg THC and 20 mg CBD.

The valerian extract may be present in an amount of 50-600 mg/dose, 100-600 mg/dose, 200-600 mg/dose, 250-500 mg/dose, or 300-400 mg/dose. Alternatively, the third active ingredient may be one or more pure valerenic acids, used in a total amount ranging from 0.1 to 1.8 mg/dose, 0.2 to 1.8 mg/dose, 0.4 to 1.8 mg/dose, 0.5 to 1.5 mg/dose, or 0.6 to 1.2 mg/dose.

Further exemplary suitable ranges within the scope of this disclosure are compositions where caffeine is replaced by another xanthine, e.g., theobromine, as seen in Table 2.

TABLE 1

| | RANGES |
|---|---|
| INGREDIENTS | mg/serving |
| Caffeine | 10-400 |
| Tetrahydrocannabinol | 1-300 |

TABLE 2

| | RANGES |
|---|---|
| INGREDIENTS | mg/serving |
| Theobromine | 10-400 |
| Tetrahydrocannabinol | 1-300 |

In an exemplary embodiment, caffeine is present in the powder composition for example in an amount of from 1 mg to 400 mg and yohimbine is present in an amount of from 1 mg to 30 mg, as shown in Table 3.

TABLE 3

| | RANGES |
|---|---|
| INGREDIENTS | mg/serving |
| Caffeine | 1-400 |
| Yohimbine | 1-30 |
| Cannabidiol | 1-300 |

The physiologically active compounds contemplated for use herein are included in the medicinal composition in an amount sufficient to produce the desired effect upon the target process, condition or disease. In addition, such compositions may optionally contain one or more agents selected from adsorbents (such as silicon dioxide), flavoring agents (such as peppermint, oil of wintergreen or cherry), coloring agents, preserving agents, and the like, in order to provide pharmaceutically elegant and palatable preparations. Suspensions of the present invention may contain wetting agents, suspending agents, buffers, preserving agents, flavors and sweeteners (sucralose, acesulfame potassium).

As described above, the medicinal composition may, for example, be in powder form. This powder composition can be prepared by:
premixing a cannabinoid oil, e.g., CBD oil or THC oil, with a carrier powder, e.g., silicon dioxide,

mixing a methylxanthine and other components in powder form in a suitable blender or planetary mixer, and

adding the premix of cannabinoid oil and the carrier powder to the other mixed components,

mixing the ingredients for the time necessary to obtain a uniform and homogeneous powdered mixture, and

adding the uniform and homogeneous powdered mixture to an aqueous carrier.

The medicinal composition may be in powder form, where the powder composition is prepared by:

mixing a cannabinoid oil, e.g., CBD oil or THC oil, and caffeine with a carrier powder,

adding the mixture of the cannabinoid oil, caffeine, and the carrier powder to an aqueous carrier to make an aqueous mixture, and then

mixing the aqueous mixture for the time necessary to obtain a uniform and homogeneous suspension.

In various embodiments, additional components may be added directly to the aqueous mixture, including extracts of various herbs. Electrolytes, e.g., sodium and/or potassium salts added to the aqueous mixture. Buffers, e.g., a mixture of phosphate salts or a sodium citrate/citric acid buffer couple, may be added to the aqueous mixture to control pH. Natural sweeteners, e.g., sugar, and/or artificial sweeteners, e.g., sucralose, may be added to the aqueous mixture, along with preservatives known in the art. In various embodiments, water-insoluble extracts of various herbs may be dispersed in the aqueous mixture or added to the carrier powder along with the cannabinoid oil and caffeine.

As described above, the instant composition may, for example, be in powder form. This powder composition can be prepared by mixing components in powder form in a suitable blender or planetary mixer and mixing the ingredients for the time necessary to obtain a uniform and homogeneous mixture.

The mixture can then optionally be packaged into jars in a quantity that represents a defined number of servings. A measuring scoop can be added to the jar for the purpose of providing an accurate serving.

The powder composition described above may be ingested orally as a powder composition. In the alternative, a user may scoop a serving of the powder from the jar and mix it with cold or room-temperature water. The powder can also be mixed with a beverage to provide a suspension. It can also be added and mixed into a fruit sauce, yogurt or pudding.

In one embodiment, the composition comprises 1 to 4.8 grams of medium chain triglycerides per 100 grams of the medicinal composition.

In one embodiment, the invention is directed to a method of providing a nootropic effect in a mammal by administering the composition described above. The mammal may, for example, be a human. The administration can, for example, be oral administration, for example by ingestion of a beverage, fruit sauce, yogurt, or pudding into which the composition described above has been mixed.

The administration may optionally be chronically for a set period-of-time, for example from two to eight weeks. As used herein, “chronically” means repeated ingestion over a period of several days, several weeks, even several months, or longer. Acute (non-chronic) administration may also be utilized. Whether chronically or non-chronic, it is meant that the composition is orally ingested one time per day, either in powder form or mixed with a beverage, fruit sauce, yogurt, pudding, or other liquid composition. The amounts given in Table 1 indicate relative amounts of each component which may be present in a single serving, said single serving optionally being ingested one or more times a day, optionally mixed with a single serving of a beverage, fruit sauce, yogurt, or pudding. The skilled artisan will understand the standard serving size of each of the above.

For example, a single serving of the powder composition as set forth above and as exemplified in Table 1 may be mixed with a single serving of yogurt, for example one cup of yogurt. The same single serving may be mixed with a single serving of pudding, for example one half cup. An exemplary serving size for water or a beverage with which the single serving powder composition may be mixed would be eight ounces.

In a specific embodiment, the subject invention provides aqueous compositions into which the powder composition has been mixed, suitable for oral administration to mammals including, without limitation, humans. To prepare a composition according to one embodiment of the invention, a desired amount of each component of the composition is added to a selected volume of an aqueous carrier, e.g., water or a beverage, and the formulation is stirred to create a dispersion of the powder composition in the aqueous carrier.

A composition according to this invention may also include other ingredients such as, for example, flavoring agents, colorants, viscosity modifiers, preservatives, chelating agents, antioxidants, surface modifiers and other medicinal adjuvant materials. Other materials include any substance that is generally recognized as promoting the health or function of a mammalian organism, including humans, or benefiting a composition useful thereof in terms of its efficacy, appearance, stability, consistency, aroma, or viscosity. Such substances include, for example, other amino acids and their salts, vitamins, minerals, fatty acids, enzymes, mono-glycerides, di-glycerides, tri-glyceride ester oils (including, for example, vegetable oils and animal fats) emulsifiers, hydrolyzed proteins, whey protein, stabilizers, flow modifiers, viscosity improvers, chelating agents, enzymes, and surfactants (whether anionic, cationic or nonionic). The total amount of these materials in a composition can be any amount between about 0.01% and about 50% by weight based on the total weight of said composition, including all percentages and ranges of percentages therebetween.

A composition according to this invention may also comprise one or more natural or synthetic beverages. For example, a natural beverage may contain the pulp, juice or any other constituent of a naturally occurring fruit, vegetable, or animal product whether from the wild, cultured, cultivated on a farm or otherwise domesticated.

Natural beverages include, without limitation, materials such as milk products, soy products, ice cream, yogurt, citrus fruit juices, non-citrus fruit juices, and vegetable juices, or components of any of the foregoing, wherein said natural beverages are present in any effective amount to impart flavor to the compositions, which may be any amount between about 0.1% and about 99% by weight based on the total weight of said composition, including all percentages and ranges of percentages there between.

Thus, it is evident that a composition according to this invention may be made quite palatable to a mammalian subject, including a human. Serving sizes may be any serving size in the range of about 10 milligrams to about 50 grams, in an aqueous solution that is from about 20 ml to about 2,500 ml in volume. Thus, for example, 10 milligrams to about 50 grams of the powder composition described above may be mixed with 20 ml to about 2,500 ml of water, juice, or other liquid composition, in particular 100 mL of water, juice, or other liquid composition. This admixing creates the oral composition which may be taken as set forth above. Such oral composition can provide a concentrate from which the required amount of each component may conveniently be provided.

The compositions of the subject invention can be formulated for a variety of modes of administration. These formulations include, but are not limited to, compositions for oral administration, emulsion compositions, gel formulations, oral solid compositions, and oral liquid compositions, or with protein.

It has been found that oral administration of the medicinal compositions according to the invention are effective provide a nootropic effect beyond the effects simple obtain by cannabinoids alone. Embodiments of the present invention would provide compositions related to the administration thereof in effectively reducing anxiety due to ingestion of xanthines containing beverages.

### Example 1

A nutraceutical beverage contains 1.5 to 2.1 mg/oz. of a cannabinoid, and 15 to 21 mg/oz. of caffeine, as follows. The cannabinoid includes 25 mg total cannabinoids, including 5 mg of THC and 20 mg of a non-psychotropic cannabinoid. The cannabinoid and the caffeine are combined with a carrier powder to form a powdered mixture, and the powdered mixture is mixed with an aqueous vehicle to form the beverage.

| Serving size | 16 oz. | 12 oz. |
|---|---|---|
| Cannabinoid (mg/serving) | 25 mg | 25 mg |
| Caffeine (mg/serving) | 250 mg | 250 mg |

### Example 2

A nutraceutical beverage contains:

0.3 to 0.42 mg/oz. of THC,

1.5 to 2.1 mg/oz. of CBD; and

15 to 21 mg/oz. of caffeine, as shown in the Table below.

THC and the non-psychotropic cannabinoid are used in a ratio of 1:5. The cannabinoid and the caffeine are combined with a carrier powder to form a powdered mixture, and the powdered mixture is mixed with an aqueous vehicle to form the beverage.

| | | |
|---|---|---|
| Serving size | 16 oz. | 12 oz. |

| | | | | |
|---|---|---|---|---|
| THC (mg/serving) | 5 | mg | 5 | mg |
| CBD (mg/serving) | 25 | mg | 25 | mg |
| Caffeine (mg/serving) | 250 | mg | 250 | mg |

### Example 2

A beverage contains 1.5 to 2.1 mg/oz. of a cannabinoid, and 15 to 21 mg/oz. of caffeine, and a valerian extract, as follows.

| Serving size | 16 oz. | 12 oz. | |
|---|---|---|---|
| Cannabinoid (mg/serving) | 25 mg | 25 | mg |
| Caffeine (mg/serving) | 250 mg | 250 | mg |
| Valerian Extract | 300 mg | 225-300 | mg |

The cannabinoid may be provided as a pure compound, such as THC, CBD, CBG, CBN, CBC, or CBDV. The cannabinoid may be the psychotropic cannabinoid THC, or a non-psychotropic cannabinoid selected from CBD, CBG, CBN, CBC, CBDV, and mixtures thereof. The cannabinoid may be a combination of psychotropic and non-psychotropic cannabinoids.

The formulation may contain extracts of arugula and/or valerian. The formulation may contain electrolytes, e.g., sodium and/or potassium. Sodium citrate and citric acid may be present as a buffer to control pH. Natural sweeteners, e.g., sugar, and/or artificial sweeteners, e.g., sucralose, may be present, along with preservatives known in the art.

All references discussed herein are incorporated by reference. One skilled in the art will readily appreciate that the present invention is well adapted to carry out the objects and obtain the ends and advantages mentioned, as well as those inherent therein. The present invention may be embodied in other specific forms without departing from the spirit or essential attributes thereof and, accordingly, reference should be made to the appended claims, rather than to the foregoing specification, as indicating the scope of the invention.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

What is claimed is:

1. A nutraceutical composition, comprising:
an aqueous carrier; and
a powder suspended in the aqueous carrier,
wherein the powder comprises a mixture of:
a first component comprising 1 to 300 mg/dose of a psychotropic cannabinoid, a non-psychotropic cannabinoid, or a mixture thereof;
a second component comprising 1 to 400 mg/dose of a methylxanthine.

2. The nutraceutical composition of claim 1, wherein said first component is selected from the group consisting of Δ9-tetrahydrocannabinol (Δ9-THC), cannabidiol (CBD), cannabigerol (CBG), Cannabinol (CBN), cannabichromene (CBC), cannabidivarin (CBDV), and mixtures thereof.

3. The nutraceutical composition of claim 1, wherein said methylxanthine is selected from the group consisting of caffeine, aminophylline, dyphylline, 3-isobutyl-1methylxanthine, paraxanthine, pentoxifylline, theobromine, theophylline, and mixtures thereof.

4. The nutraceutical composition of claim 1, wherein said methylxanthine is selected from the group consisting of caffeine, aminophylline, dyphylline, 3-isobutyl-1methylxanthine, paraxanthine, pentoxifylline, and mixtures thereof.

5. The nutraceutical composition of claim 1, further comprising an extract of valerian containing a valerenic acid selected from the group consisting of valerenic acid, hydroxyvalerenic acid, acetoxyvalerenic acid, and mixtures thereof.

6. The nutraceutical composition of claim 1, wherein said composition is in the form of an orally administrable liquid composition.

7. The nutraceutical composition of claim 6, wherein said orally administrable liquid composition comprises water, fruit juice, yogurt, or pudding.

8. The nutraceutical composition of claim 6, wherein said composition is in the form of a dispersion or suspension of a powder in an aqueous medium.

9. A nutraceutical composition, comprising:
a first component comprising 2 to 50 mg/dose of a psychotropic cannabinoid, a non-psychotropic cannabinoid, or a mixture thereof;
a second component comprising 80 to 300 mg/dose of a methylxanthine; and
an aqueous carrier,
wherein the nutraceutical composition is free of non-cannabinoid active components of Cannabis sattiva and the nutraceutical composition contains no active components of coffee other than the methylxanthine.

10. The nutraceutical composition of claim 9, wherein the first component and the second component are used in a ratio from 1:5 to 1:12.

11. A method comprising:
administering an effective amount of a nutraceutical composition to a mammal in need thereof,
wherein the nutraceutical composition comprises:
a first component comprising 1 to 300 mg/dose of a psychotropic cannabinoid, a non-psychotropic cannabinoid, or a mixture thereof; and
a second component comprising 1 to 400 mg/dose of a methylxanthine; and
imparting a nootropic effect beyond effects obtained by the first component alone.

12. A method comprising:
administering an effective amount of a nutraceutical composition to a mammal in need thereof, wherein the nutraceutical composition comprises
a first component comprising 10 to 50 mg/dose of a psychotropic cannabinoid, a non-psychotropic cannabinoid, or a mixture thereof; and
a second component comprising 80 to 300 mg/dose of a methylxanthine; and
imparting a nootropic effect beyond effects obtained by the first component alone.

13. The method of claim 11, wherein said administering is once a day over a period of treatment of one—eight weeks.

14. The method of claim 12, wherein said administering is once a day over a period of treatment of one—eight weeks.

15. The method of claim 11, wherein the method includes, prior to said administering,
providing said nutraceutical composition in a powder form;
dispersing or suspending the nutraceutical composition in an aqueous vehicle or carrier to form a liquid composition.

16. The method of claim 12, wherein the method includes, prior to said administering,
providing said nutraceutical composition in a powder form;
dispersing or suspending the nutraceutical composition in an aqueous vehicle or carrier to form a liquid composition.

17. The nutraceutical composition of claim 1, wherein the composition is in the form of a powder.

18. The nutraceutical composition of claim 9, wherein the composition is in the form of a powder.

19. The nutraceutical composition of claim 9, further comprising an extract of valerian or arugula.
